(19) United States
(12) Patent Application Publication
(10) Pub. No.: US 2008/0262390 A1
(43) Pub. Date: Oct. 23, 2008

(54) Fiducials for placement of tissue closures

(75) Inventors: Mahalaxmi Gita Bangera, Renton, WA (US); Edward S. Boyden, Cambridge, MA (US); Roderick A. Hyde, Redmond, WA (US); Muriel Y. Ishikawa, Livermore, CA (US); Edward K.Y. Jung, Bellevue, WA (US); Eric C. Leuthardt, St. Louis, MO (US); Dennis J. Rivet, St. Louis, MO (US); Michael A. Smith, Phoenix, AZ (US); Elizabeth A. Sweeney, Seattle, WA (US); Clarence T. Tegreene, Bellevue, WA (US); Lowell L. Wood, Bellevue, WA (US); Victoria Y.H. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/880,478

(22) Filed: Jul. 20, 2007

Related U.S. Application Data
(63) Continuation-in-part of application No. 11/788,767, filed on Apr. 19, 2007.

(51) Int. Cl. A61B 17/00; A61B 18/18

(52) U.S. Cl. 600/587; 128/897; 128/898; 606/1; 606/33

(57) ABSTRACT

Fiducial markings are placed on a patient to identify surgical parameters such as locations for an incision or a surgical closure.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. 11/788,767, entitled SYSTEMS AND METHODS FOR APPROXIMATING SURFACES, naming MAHALAXMI GITA BANGERA, EDWARD S. BOYDEN, RODERICK A. HYDE, MURIEL Y. ISHIKAWA, EDWARD K. Y. JUNG, ERIC C. LEUTHARDT, DENNIS J. RIVET II, MICHAEL A. SMITH, ELIZABETH A. SWEENEY, CLARENCE T. TEGREENE, LOWELL L. WOOD, JR., VICTORIA Y. H. WOOD as inventors, filed Apr. 19, 2007, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 11/805,719, entitled SYSTEMS AND METHODS FOR APPROXIMATING SURFACES, naming MAHALAXMI GITA BANGERA, EDWARD S. BOYDEN, RODERICK A. HYDE, MURIEL Y. ISHIKAWA, EDWARD K. Y. JUNG, ERIC C. LEUTHARDT, DENNIS J. RIVET, MICHAEL A. SMITH, ELIZABETH A. SWEENEY, CLARENCE T. TEGREENE, LOWELL L. WOOD, JR., AND VICTORIA Y. H. WOOD as inventors, filed May 23, 2007, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 11/811,885, entitled SYSTEMS AND METHODS FOR CLOSING OF FASCIA, naming MAHALAXMI GITA BANGERA, EDWARD S. BOYDEN, RODERICK A. HYDE, MURIEL Y. ISHIKAWA, EDWARD K. Y. JUNG, ERIC C. LEUTHARDT, ELIZABETH E. NUGENT, DENNIS J. RIVET, MICHAEL A. SMITH, ELIZABETH A. SWEENEY, CLARENCE T. TEGREENE, LOWELL L. WOOD, JR., VICTORIA Y. H. WOOD as inventors, filed Jun. 11, 2007, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### SUMMARY

In one aspect, a system for planning placement of tissue anchors during a surgery includes input circuitry configured to receive a signal including information relating to a planned surgery on the body of an animal (e.g., a mammal or a human), and anchor placement circuitry configured to use the received signal to determine preferred placement of tissue anchors for surgery. The input circuitry may include a sensor configured to measure a physiological parameter of the animal, such as the geometry, location, size, or shape of a physiological structure, or a fiducial marking a planned incision location on the animal. The signal may include an identifier for the animal, and identifier for the surgery type, or a physiological parameter of the animal (e.g., a geometric measurement of a physiological structure). The anchor placement circuitry may include tissue modeling circuitry, which may be configured to build a virtual model of at least a portion of the body, and may further be configured to calculate expected tissue response to a selected tissue anchor configuration or to apply a heuristic rule to determine a tissue anchor configuration. The anchor placement circuitry may include stress estimation circuitry, which may be configured to determine expected stresses on tissue anchors or on tissue in a selected tissue anchor configuration, or may include optimizing circuitry configured to determine an optimum anchor configuration for a specified design goal. The anchor placement circuitry may include an anchor placement pattern library, which may include stored configurations of tissue anchors, which may be specified by an operator or previously calculated. The anchor placement circuitry may include anchor form factor selection circuitry, which may include stored information about the form factors of a plurality of tissue anchors, stored information about available sizes of tissue anchors, stored information about mechanical properties of tissue anchors, or circuitry configured to select a suggested anchor configuration for the planned surgery. The system may further include an output configured to display the determined preferred placement of tissue anchors (e.g., as a machine-readable output or as a human-readable output). The output may include a patient marking device, which may be configured to mark the determined preferred placement of tissue anchors on the patient or on a stabilizing member configured to be attached to the patient, or which may be configured to place tissue anchors on the patient in accordance with the determined preferred placement of tissue anchors. The output may include a printer that marks a tape with fiducial marks indicating the preferred placement of tissue anchors, wherein the tape is configured to be attached to the body of the animal (e.g., by an adhesive layer).

In another aspect, a tool for placement of fiducial marks on a body of an animal (e.g., a mammal or a human) includes a marking roller configured to be rolled along the surface of the body of the animal, the marking roller including a fiducial-marking surface configured to make at least one fiducial mark on the surface of the body as the fiducial-marking surface comes in contact with it. The at least one fiducial mark indicates locations for a surgical incision and for a surgical closure element. The at least one fiducial mark may include human-readable or computer-readable information. The at least one fiducial mark may include a substantially continuous line along a path followed by the roller, which may indicate the planned location of the surgical incision. The at least one fiducial mark may include an alignment mark offset from the substantially continuous lane, which may indicate the planned location of a surgical closure element. The roller may include a cutting edge configured to cut tissue along a cutting path, which may indicate the location of the surgical incision. The roller may be configured to make an alignment mark substantially contemporaneously with cutting, which may indicate the planned location of a surgical closure element. The tool may be configured to make the at least one fiducial mark on the animal's skin or on a tissue selected from the group consisting of bone, muscle, organs, and connective tissue.

In yet another aspect, a surgical tool for placement of fiducial marks on a body of an animal (e.g., a mammal or a human) includes a tape dispenser configured to dispense a tape configured for attachment to the body of the animal, wherein the tape includes at least one fiducial mark thereon that indicates a surgical plan. The tape may include an adhesive layer configured to attach the tape to the body of the animal, and may include an opening for an incision. The at least one fiducial mark may identify an incision location, and may be machine-readable or human-readable. The tape dispense may include a roller loaded with the marked tape, which may be configured to allow a user to see a body surface as the tape is dispensed from the roller onto the body surface. The tool may further include a printer configured to place the at least one fiducial mark on the tape. The tape may be configured for attachment to the animal's skin, or to a tissue selected from the group consisting of bone, muscle, organs, and connective tissue.

In still another aspect, a surgical tool for placement of fiducial marks on the body of an animal (e.g., a mammal or a human) includes an energy source configured to induce a modification of an energy-responsive material on the body, wherein the modification forms at least one fiducial mark indicative of a surgical plan. The at least one fiducial mark may indicate an incision location or the location of a surgical closure element, and may be human-readable or machine-readable. The tool may further include identification circuitry and controller circuitry. The identification circuitry is configured to identify an incision location for a surgical closure, and determine an incision mark location for the at least one fiducial mark that characterizes the incision location for the surgical closure. The controlled circuitry is configured to direct the energy source to modify the energy-responsive material in accordance with the determined incision mark location to form the at least one fiducial mark. The identification circuitry may be further configured to identify an alignment location for a surgical closure element, and to determine an alignment mark location for the at least one fiducial mark that characterized the identified alignment location, and the controller circuitry may be further configured to direct the energy source to modify the energy-responsive material in accordance with the determined alignment mark location to form the at least one fiducial mark. The tool may further include a beam-directing element (e.g., a lens, a splitter, a mask, a polarizer, a mirror, an electromagnetic field, a diffractive element, or a refractive element) interposed between the energy source and the body. The energy source may produce electromagnetic energy (e.g., a laser or an X-ray source) or may produce a particle beam (e.g., an ion beam or an electron beam). The energy-responsive material may be disposed on the skin of the body, or on a tissue of the body selected from the group consisting of bone, muscle, organs, and connective tissue.

In yet still another aspect, a method of preparing a patient (e.g., a mammal or a human) for surgery includes identifying at least one preferred attachment point for a surgical closure, and placing at least one fiducial mark at each of the at least one preferred attachment points, wherein the at least one fiducial mark is machine-readable. Placing the at least one fiducial mark may include placing the at least one fiducial mark with a marking roller (in which case the method may further include constructing the marking roller in a configuration to place the at least one fiducial mark at each of the at least one preferred attachment points), adhering a stabilizing member including the at least one fiducial mark to the patient, or applying an energy-sensitive material to the patient and exposing the energy-sensitive material to a spatially-patterned energy flux that modifies the energy-sensitive material to produce the at least one fiducial mark. Identifying at least one preferred attachment point for a surgical closure may include measuring a physiological parameter of the patient, constructing a virtual model of at least a portion of the patient (and optionally further using the virtual model to calculate expected tissue response to placement of a surgical closure at the at least one preferred attachment point), determining expected stresses on a surgical closure at the at least one preferred attachment point, or selecting a surgical closure configuration from a pattern library. The at least one fiducial mark may include three-dimensional surgical information. Placing the at least one fiducial mark may include placing the at least one fiducial mark on the patient's skin, or on a tissue of the patient selected from the group consisting of bone, muscle, organs, and connective tissue.

In a further aspect, a method of preparing a patient (e.g., a mammal or a human) for surgery includes identifying at least one preferred attachment point for a surgical closure, and placing at least one fiducial mark on a stabilizing member configured to be adhered to the patient, where the at least one fiducial mark is placed to indicate the at least one preferred attachment point upon adhering the stabilizing member to the patient. The method may further include adhering the stabilizing member to the patient (e.g., by loading the stabilizing member into a dispenser and dispensing the stabilizing member from the dispenser), for example to the patient's skin or to a tissue selected from the group consisting of bone, muscle, organs, and connective tissue. Identifying the at least one preferred attachment point for a surgical closure may include measuring a physiological parameter of the patient, constructing a virtual model of at least a portion of the patient (and optionally further using the virtual model to calculate expected tissue response to placement of a surgical closure at the at least one preferred attachment point), determining expected stresses on a surgical closure at the at least one preferred attachment point, or selecting a surgical closure configuration from a pattern library. Placing the at least one fiducial mark on the stabilizing member may include printing the at least one fiducial mark on the stabilizing member. The stabilizing member may include at least one registration mark configured to identify a location for placement of the stabilizing member on the patient, at least one label identifying the at least one fiducial mark, or identifying information for the patient. The at least one fiducial mark may include three-dimensional surgical information.

In still a further aspect, a method of preparing the body of a patient (e.g., a mammal or a human) for surgery includes identifying at least one preferred attachment point for a surgical closure, applying an energy-responsive material to the body, and exposing the energy-responsive material to a spatially patterned energy flux, the spatially patterned energy flux modifying the energy-responsive material to form at least one fiducial mark placed to indicate the at least one preferred attachment point for the surgical closure. Identifying the at least one preferred attachment point for a surgical closure may include measuring a physiological parameter of the patient, constructing a virtual model of at least a portion of the patient (and optionally further using the virtual model to calculate expected tissue response to placement of a surgical closure at the at least one preferred attachment point), determining expected stresses on a surgical closure at the at least one preferred attachment point, or selecting a surgical closure configuration from a pattern library. The spatially patterned energy flux may be electromagnetic radiation (e.g., generated by a laser). The at least one fiducial mark may include three-dimensional information. Applying the energy-responsive material to the body may include applying it to the patient's skin, or to a tissue selected from the group consisting of bone, muscle, organs, and connective tissue.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic of an incision being closed with a suture and a set of tissue anchors.

FIG. 2 is a schematic of several different anchor embodiments.

FIG. 3 is a schematic of an incision being closed with a securing member and a set of tissue anchors.

FIG. 4 is a schematic of anchors arranged on a stabilizing member.

FIG. 5 is a schematic of several different embodiments of multi-part couplers.

FIG. 6 is a schematic of several different multi-part anchor embodiments.

FIG. 7 is a schematic of a two-part trocar for use in closing the fascia in a laparoscopic procedure.

FIG. 8 is a schematic of fascia being closed with a suture and a set of tissue anchors.

FIG. 9 is a schematic of a portion of another two-part trocar for use in a laparoscopic procedure.

FIG. 10 is a schematic of a single-tube trocar with an openable port for use in a laparoscopic procedure.

FIG. 11 is a schematic of a single-tube trocar with tissue anchors on its exterior.

FIG. 12 is a flow chart of a method of closing a wound.

FIG. 13 is a flow chart of a method of performing surgery.

FIG. 14 is a flow chart of a method of preparing a body for surgery.

FIG. 15 is a schematic of a computer-implemented system for determining placement of tissue anchors.

FIG. 16 is a schematic of a marking roller for placing fiducial marks on a body.

FIG. 17 is a schematic of a dispenser for a body-adherent tape.

FIG. 18 is a schematic of a tape suitable for loading in the dispenser of FIG. 17, the tape including fiducial marks in accordance with a surgical plan.

FIG. 19 is a schematic of a laser marking system for marking a body in preparation for surgery.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

As used herein, the term “biocompatible” means a material the body generally accepts without a significant immune response/rejection or excessive fibrosis. In some embodiments, some immune response or fibrosis is desired. In other embodiments, vascularization is desired. In still other embodiments, vascularization is not desired. Biocompatible materials include, but are not limited to, synthetic organic materials such as clinically used nonbiodegradable and biodegradable and bioresorbable polymers including polyglycolide, optically active and racemic polylactides, polydioxanone, and polycaprolactone, polymers under clinical investigation including polyorthoester, polyanhydrides, and polyhydroxyalkanoate, early stage polymeric biomaterials including poly(lactic acid-co-lysine), and shape memory polymers (e.g., block copolymers of oligo(ε-caprolactone)diol and crystallisable oligo(ρ-dioxanone)diol, as described in Lendlein, et al., “Biodegradable, elastic shape-memory polymers for potential biomedical applications,” Science, 296(5573):1673-1676 (2002), which is incorporated by reference herein).

As used herein, “biodegradable” materials include materials that at least partially resorb into the body or otherwise break down over time, while “nonbiodegradable” materials include those that maintain substantial mechanical integrity over their lifetime in a body. Such “biodegradable” or “nonbiodegradable” materials are well known to those having skill in the art. In general, the anchors, couplers, traction members, securing members, tensioning members, stabilizing members, and other components described herein may be either biodegradable or nonbiodegradable, or may include both biodegradable and nonbiodegradable components. In some embodiments, these elements will be biocompatible, while in other embodiments, they may be partially or fully constructed from nonbiocompatible materials.

As used herein, “antimicrobial” materials include materials that have the capacity to inhibit the growth of or destroy pathogens, including but not limited to bacteria, fungi, and viruses. Such antimicrobial materials are well known to those having skill in the art and may include materials that are coated or impregnated with an antimicrobial agent or wherein the material itself possesses antimicrobial properties.

As used herein, a material having a “therapeutic property” is one that induces or facilitates a desired biological response. Materials having a therapeutic property are well know to those having skill in the art, and include, but are not limited to cell growth promoters, cell growth inhibitors, cytokines, healing promoters, antibiotics, clotting modulators, anti-inflammatories, and anti-scarring agents.

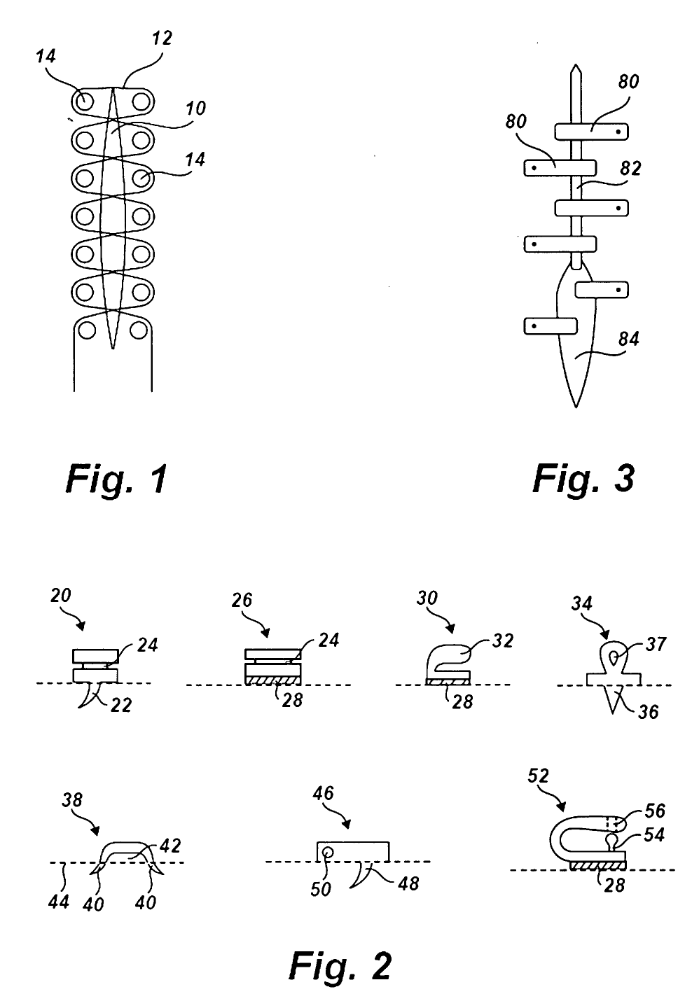

FIG. 1 illustrates an incision 10 being closed with a suture 12 and a set of tissue anchors 14. The tissue anchors 14 may be placed in the tissue before or after the incision 10 is made, and may be shaped to receive the suture 12. To close the incision 10, the suture 12 is wound around the anchors 14 as shown and pulled to tighten, approximating the body tissue in the region of the anchors 14. In some embodiments, the edges of the incision may be brought together by other means (e.g., manually by the surgeon), and the suture 12 may be used to maintain the approximation of the edges of the incision. Those of skill in the art of surgery will recognize that there are many possible patterns for placement of the anchors 14 and for winding of the suture 12, and will be able to select an appropriate configuration for any particular patient and incision. For example, the crossed suture 12 shown in FIG. 1 may not be desirable in all cases, and may be replaced by a suture winding that does not cross itself, such as a configuration (not shown) in which discrete sutures draw anchors 14 together pairwise across the incision 10, or a single suture arranged in a serpentine pattern. In other embodiments, it may not be desirable to place all anchors 14 at the same distance from incision 10, or to place the anchors 14 at regular intervals as shown in FIG. 1. For example, an irregular pattern or a pattern with localized concentrations of anchors 14 may be appropriate for locations having differential topographies, tissue types, expected movement ranges, stresses, or contact with surfaces, such as bandages, supports, clothing, or similar. The number and placement of the anchors 14 will also vary with the incision type, with fewer anchors 14 typically (but not always) being applied for smaller incisions. While FIG. 1 illustrates an incision 10 being closed, a similar arrangement may be used to close an accidental wound or to draw tissue into a desired configuration (e.g., in a face lift or other cosmetic procedure, or in a bladder suspension), or to attach tissue to an implanted device or other object (e.g., an organ for transplant) in a body. While FIG. 1 illustrates a straight incision 10, in other embodiments, the opening to be closed by the anchors may be curved, round, branched, stellate, or angled (e.g., in a sawtooth configuration).

FIG. 2 shows a variety of anchor configurations that may be used with a suture to close an incision as shown in FIG. 1. Anchor 20 includes a piercing structure 22 for placement in a body tissue, and a groove 24 to receive a suture. Anchor 26 also includes a groove 24 to receive a suture, but is adhered to the tissue via an adhesive layer 28. Anchor 30 is adhered to the tissue with an adhesive layer, and includes a hook 32 about which a suture may be looped. Anchor 34 includes a piercing structure 36 of a slightly different shape from that of anchor 20, and also includes an eyelet 37 through which a suture may be threaded. The piercing structure 36 may allow the anchor to be rotated, either manually or through the natural pulling action of a threaded suture. Anchor 38 includes two piercing prongs 40 like a staple, and creates an opening 42 through which a suture may be passed in cooperation with the underlying tissue 44. In some embodiments, this anchor may be pushed further into the tissue in a way that prevents movement of the suture, for example after the incision has been closed. Anchor 46 includes a piercing structure 48 and an eyelet 50, the eyelet 50 being disposed distal from the piercing structure 48 and along the surface of the body tissue. In some embodiments, the eyelets 50 of adjacent anchors 46 may be aligned as the tissue is closed. Anchor 52 includes a vertical post 54 and channel 56 allowing it to be snapped closed, for example after a suture has been threaded around it. In some embodiments, this closure may be reversible, while in others, it may be irreversible. In some embodiments, closure of the anchor 52 may restrict sliding of the suture, while in other embodiments, the suture may be able to slide through the anchor 52 after closure.

The specific structures of anchors shown in FIG. 2 shall not be interpreted to limit the shape or design of the anchors described and claimed herein. By way of non-limiting example, the piercing structure 22 of anchor 20 may be used in place of the adhesive layer 28 shown with anchor 30; the eyelet 50 of anchor 46 may be used with the adhesive layer 28 shown with anchor 30 or with the piercing structure 36 shown with anchor 34. Various combinations of the piercing structures shown, as well as those not shown but known to those of skill in the art, can be used with any suture-holding structure or any other securing member or mechanism.

FIG. 3 shows another embodiment, in which anchors 80 are secured via insertion of a securing member 82, which is a conformable rod in the illustrated embodiment. The illustrated anchors 80 are similar to the anchors 46 of FIG. 2, including a piercing structure (not visible in FIG. 3) and an eyelet through which conformable rod 82 may be inserted. As shown, the rod 82 is partially inserted through the anchors 80, so that the incision 84 is partially closed. In other embodiments, the anchors 80 or the securing member 82 may include other attachment structures, such as hooks, mating surfaces (to which adhesive may optionally be applied), or mechanical fasteners (e.g., hook and loop fasteners, draw latches, screws, etc.). By way of non-limiting example, securing member 82 may include a series of hooks configured to receive anchors 80; anchors 80 may include hooks configured to attach to securing member 82 (which may optionally include predetermined attachment points for anchors 80); securing member 82 may include snap fittings into which mating portions of anchors 80 may be inserted; anchors 80 and securing member 82 may include holes or other areas configured for attachment of screws or other fasteners that secure anchors 80 and securing member 82 together. In embodiments in which the securing member 82 is conformable, it may be conformed to match the shape of an incision, or it may be conformed before or after insertion in order to apply a mechanical force to tissue in order to reshape it (e.g., in cosmetic surgery). In some embodiments, the process of inserting securing member 82 may bring the anchors 80 together, while in other embodiments, the edges of the incision 84 may be brought into alignment before the securing member 82 is deployed.

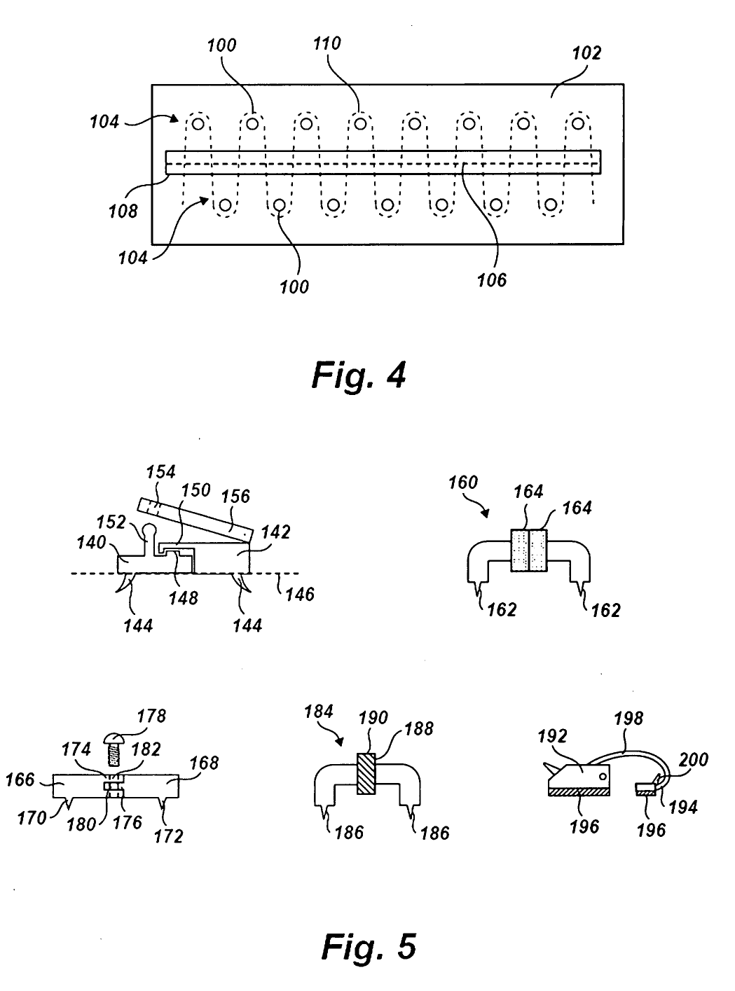

FIG. 4 is a schematic of anchors 100 arranged on a stabilizing member 102. In the embodiment shown, the stabilizing member 102 includes a flexible tape base designed to adhere to the tissue of interest. The anchors are arranged in parallel rows 104 on opposite sides of a planned incision site 106. In some embodiments, the flexible tape base may be placed on the patient prior to making the incision. The illustrated embodiment includes an opening 108 along the planned incision site 106, but other embodiments may omit the opening. The anchors 100 may adhere to the stabilizing member 102, which in turn adheres to the tissue of interest, via an adhesive, or they may include mechanical fasteners or other structures to facilitate their attachment to tissue (e.g., piercing structures such as those shown in anchors 20, 34, 38, 46 of FIG. 2). In one method of use, the stabilizing member 102 is placed on the body with opening 108 positioned at the planned incision site 106. The incision is made, and surgery is performed on the body via the incision. At the conclusion of the surgery, a suture is threaded around the anchors 100 along serpentine path 110, and tightened to draw the anchors 100 together, thereby closing the incision (in some embodiments, the incision may be closed by other means, and the suture may maintain the closure). In other embodiments, opening 108 may be omitted, and the incision performed through the stabilizing member 102, or the stabilizing member 102 may be placed after the incision is made (e.g., after the surgery is completed). In some embodiments, the stabilizing member 102 may be applied to a wound (e.g., an accidental wound). Rather than a suture, the incision may be closed by application of a securing member as described herein in connection with FIG. 3, or by direct connection of couplers as described below in connection with FIG. 5. The stabilizing member 102 may be placed on the skin, or on other tissue such as muscular or vascular tissue.

FIG. 5 shows several different embodiments of couplers that may be connected without the use of a tensioning member or a securing member as described herein. In some embodiments, a specialized or general purpose tool may be used to connect anchors together. Couplers 140, 142 include piercing structures 144 that secure the couplers to underlying tissue 146. Coupler 140 includes a temporary alignment pin 148 configured to mate with a corresponding alignment groove 150 on coupler 142. In addition, coupler 140 includes a permanent (or, optionally, semipermanent) retaining pin 152 configured to mate with a channel 154 in hinged connector 156 on coupler 142. In one method of use, the couplers 140, 142 may be secured to tissue with temporary alignment structures 148, 150 connected. The temporary alignment structures 148, 150 may then be disconnected to permit access to an incision site, for example to open an incision after the couplers 140, 142 have been placed. Upon closing, both temporary alignment structures 148, 150 and permanent retaining structures 152, 154 may be connected, permanently (or, optionally, semipermanently) closing the incision while maintaining the alignment of underlying tissue.

Couplers 160 include piercing structures 162, and permanent magnets 164. In use, these couplers may be placed on either side of a wound or a planned incision, and optionally rotated to increase the distance between permanent magnets 164 during access to the wound. Upon closing, the couplers 160 may be rotated (if necessary) to align the magnets, and brought into proximity to magnetically adhere them together, securing the underlying tissue. Couplers 166, 168 include piercing structures 170, 172 for securing them to tissue. A groove 174 in coupler 166 mates with a tongue 176 in coupler 168 to couple the couplers. This connection can be reversibly or irreversibly secured by insertion of a screw 178 through channels 180, 182 in the couplers 166, 168. Couplers 184 include piercing structures 186, and matable surfaces 188. In use, these couplers may be placed on either side of a wound or a planned incision, and optionally rotated to orient the matable surfaces away from the work area. Upon closing, the couplers 184 may be rotated (if necessary) to align the matable surfaces, which may then be secured together with adhesive 190. Couplers 192, 194 include adhesive 196 for attachment to tissue (or to a stabilizing member, not shown, or other mechanism for attachment to tissue). Coupler 192 includes latch arm 198, which engages keeper 200 on coupler 194 to form a draw latch assembly. Latch arm 198 may be rotated away from the work area during surgery, and subsequently engaged to close an underlying incision.

While the couplers illustrated in FIG. 5 are generally illustrated for coupling in pairwise configurations, in other embodiments, couplers may cooperate in larger groups to close incisions or other wounds. For example, couplers may be arranged in a “zipper” configuration to close a wound along its length. Such an arrangement may include a specialized or general-purpose coupling tool (e.g., a zipper pull) to connect couplers together or to separate them.

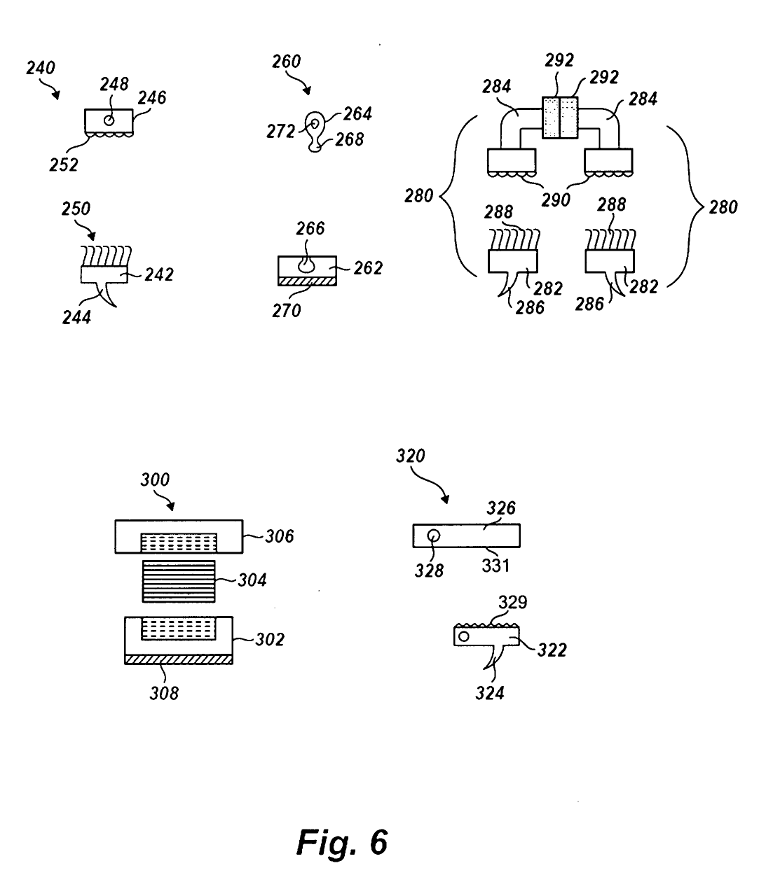

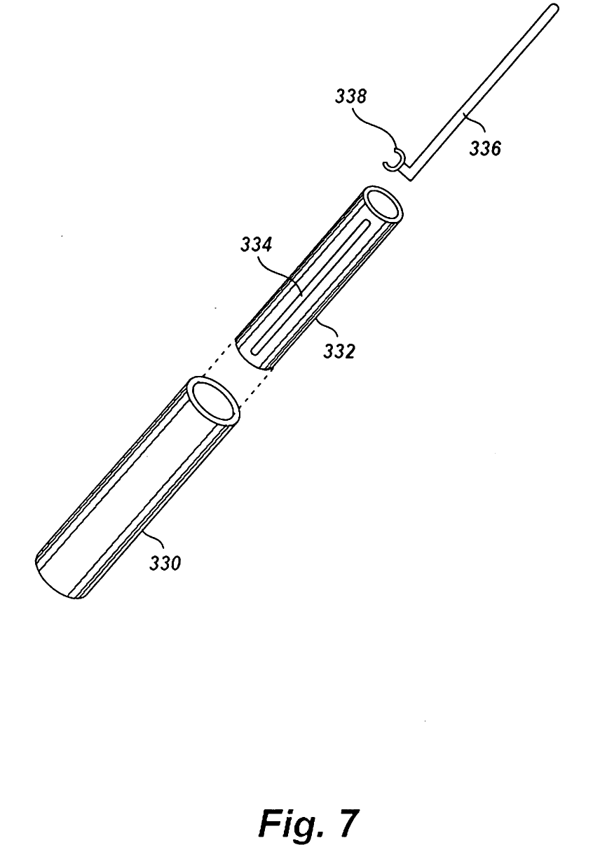

FIG. 6 is a schematic of several different multi-part anchor embodiments. Each embodiment includes a portion that adheres to tissue, and a portion that engages a suture, a stabilizing member, another anchor, or another closing mechanism. Anchor 240 includes a tissue adherent portion 242, which is configured to adhere to tissue via piercing mechanism 244, and connector portion 246, which is configured to engage a suture via opening 248. The tissue adherent portion 242 and the connector portion 246 are configured to be connected together via hook-and-loop fasteners 250, 252 (e.g., VELCRO™). Anchor 260 includes a tissue adherent portion 262 and a connector portion 264, which are configured to snap together via mechanical fasteners 266, 268. Tissue adherent portion 262 includes an adhesive layer 270 configured to adhere to tissue. Connector 264 includes an eyelet 272 configured to receive a suture (not shown). In some embodiments, mechanical fasteners 266, 268 may be configured to form a rotatable connection, which may facilitate alignment of a suture. In either embodiment of anchors 240 or 260, connector portions 246 or 264 may optionally be pre-threaded onto a suture or a stabilizing member before they are connected to their respective tissue adherent portions 242 or 262, or they may be connected to their respective tissue adherent portions 242 or 262 and subsequently threaded with a suture or stabilizing member.

Anchors 280 each include a tissue adherent portion 282 and a connector portion 284. The tissue adherent portions 282 are configured to adhere to tissue via piercing structures 286. Connector portions 284 are configured to attach to tissue adherent portions 282 via hook-and-loop fasteners 288 and 290 (e.g., VELCRO™). Connector portions 284 are also configured to engage one another via magnets 292. In one method of use, tissue adherent portions 282 may be placed on opposing sides of an incision site, before or after cutting the incision. Upon closing, connectors 284 may be connected to tissue adherent portions 282 and their respective magnets 292 engaged (before or after connection to tissue adherent portions 282), thereby closing the incision.

Anchor 300 is a three-part anchor, including a tissue adherent portion 302, a first connector portion 304 configured to screw into tissue adherent portion 302, and a second connector portion 306 configured to screw onto connector portion 304. In one method of use, a plurality of tissue adherent portions 302 are adhered to tissue via adhesive layers 308, for example before an incision is made in the tissue. When it is desired to close the opening, first connector portions 304 are screwed into each respective tissue adherent portion 302. At this point, a suture or other tensioning member (not shown) may be wound about connector portions 304. In other embodiments, second connector portions 306 may be partially or fully screwed onto their respective first connector portions 304 before winding or before tightening of the tensioning member. In some embodiments, once the tensioning member has been tightened sufficiently to close the incision, second connector portions 306 may be further screwed onto first connector portions 304, thereby clamping the tensioning member between tissue adherent portions 302 and second connector portions 306, thereby inhibiting further movement of the tensioning member.

Anchor 320 includes tissue adherent portion 322, which adheres to tissue via piercing structure 324, and connector portion 326, which includes eyelet 328. Tissue adherent portion 322 and connector portion 326 are configured to attach to one another via van der Waals forces. In the illustrated embodiment, surface 329 includes nanotubes that adhere to flat surface 331 when they are placed in contact (see, e.g., Yurdumakan, et al., “Synthetic gecko foot-hairs from multiwalled carbon nanotubes,” Chem. Commun., 2005:3799-3801, which is incorporated by reference herein). In this embodiment, eyelet 328 is located at a distal end of tissue adherent portion 322 when tissue adherent portion 322 and connector portion 326 are attached together. In some embodiments, a straight (or shaped) stabilizing element (not shown) may be threaded through eyelets 328 of a plurality of anchors 320 on opposing sides of a wound, for example in the configuration illustrated in FIG. 3.

FIG. 7 illustrates a two-part trocar for use in laparoscopic procedures. The trocar includes a first cylinder 330 having solid walls, and a second cylinder 332 having one or more longitudinal slots 334. As shown, the second cylinder 332 is sized to fit snugly within first cylinder 330. In other embodiments, the outer diameter of second cylinder 332 may be smaller than the inner diameter of first cylinder 330, producing a loose fit between the cylinders. In still other embodiments, the second cylinder 332 may be sized to fit over first cylinder 330, with either a loose or a snug fit. In still other embodiments, the first cylinder 330 may be eliminated. In such embodiments, if it is necessary to insufflate the underlying body cavity, it may be desirable that a mechanism for sealing slots 334 be integrated into second cylinder 332 in order to maintain pressure within the cavity.

In one method of use, first cylinder 330 is inserted into a body cavity (e.g., the abdominal cavity), using a round cutter (not shown) to penetrate the cavity wall. Second cylinder 332 may be integral with first cylinder 330 during insertion, or may be inserted into (or around) first cylinder 330 previously or subsequently, either before or after a laparoscopic procedure is performed. For example, the first cylinder 330 may be inserted as a conventional trocar, and a laparoscopic procedure may be performed. Subsequent to the procedure, but before closing, second cylinder 332 is then inserted into first cylinder 330, and first cylinder 330 is fully or partially retracted from the body. An anchor placement device 336, loaded with anchor 338 is then inserted into second cylinder 332. As shown, the anchor is a split ring, but any of the anchor configurations described herein may be used. In the illustrated embodiment, anchor 338 includes a shape memory alloy. The anchor 338 is inserted through the slot 334 to contact opposing sides of the fascia, and the shape memory phase change is triggered (e.g., by local heating), closing the split ring and piercing the fascia. Multiple anchors 338 may be placed, either using multiple slots 334 or by rotating second cylinder 332 in order to access different positions along the circumference of the fascial opening. Once the anchors 338 have been placed, second cylinder 332 may be fully or partially withdrawn from the opening.

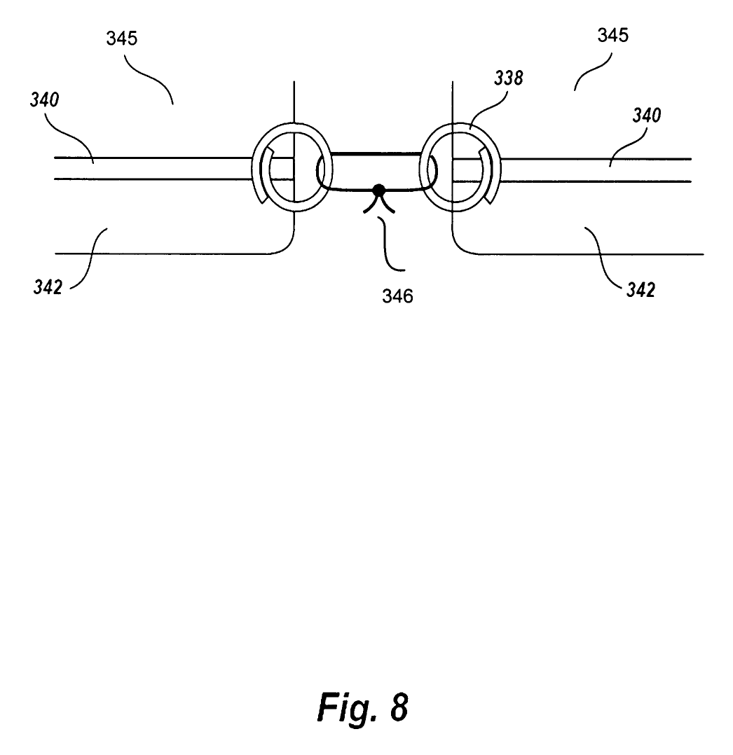

FIG. 8 illustrates two split ring anchors 338 which have pierced the fascia 340 on either side of a round laparoscopic incision. As shown, the anchors 338 also at least partially penetrate peritoneum 342 and fatty tissue 345. Anchors 338 are connected by a suture 346. The suture may be threaded before or after removal of cylinders 330, 332. Tension may be applied to suture 346 to close the fascia, for example after cylinders 330, 332 have been removed from the incision. In the illustrated embodiment, the suture connects two anchors 338 on opposing sides of the incision, but it will be understood that more anchors may be connected, either by a single suture or other connector looped through all of them, or by a series of pairwise connections (or other connections of smaller subsets of the placed anchors). Tissue anchors may be analogously used to close other layers such as the peritoneum, the muscle layers, or the skin. While split-ring anchors 338 have been illustrated in FIG. 7 and FIG. 8, other anchor configurations may be more or less desirable for any particular tissue type and geometry. For example, a shape-memory surgical staple such as those described in U.S. Pat. Nos. 4,485,816 and 6,133,611 (both of which are incorporated by reference herein) may be used as a tissue anchor for some surgeries. In some configurations, a suture or other tensioning device may be prethreaded onto tissue anchors, or the anchors may be configured to couple to one another without use of a tensioning device.

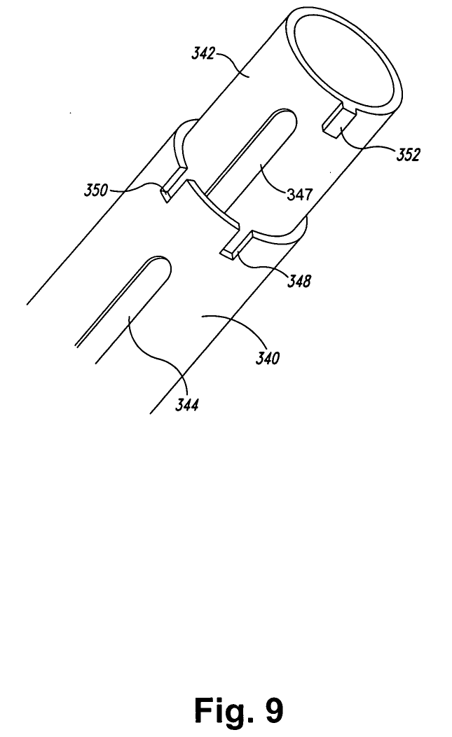

FIG. 9 illustrates a proximal end of another two-part trocar for use in laparoscopic procedures. The illustrated trochar includes two concentric cylindrical members 340, 342, each of which includes a longitudinal slot 344, 347. The outer cylinder 340 includes two notches 348, 350, which are each configured to engage a tab 352 on the inner cylinder 342. During insufflation, tab 352 is engaged with notch 350. In this configuration, slots 344 and 347 are not aligned with one another, so that insufflation gas does not leak from the trochar through the slots. When it is desired to access the fascia, tab 352 is disengaged from notch 350 by partially withdrawing inner cylinder 342, as shown, and the cylinders are relatively rotated to align tab 352 with notch 348. In this configuration, slots 344 and 347 are aligned with one another, so that the fascia may be accessed through the side of the trocar. In some embodiments, the cylinders may be transparent in at least the region of the slots, for example to aid the surgeon in visualizing the fascia.

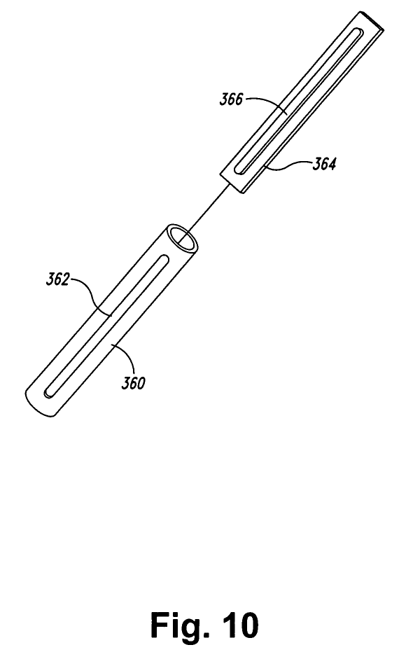

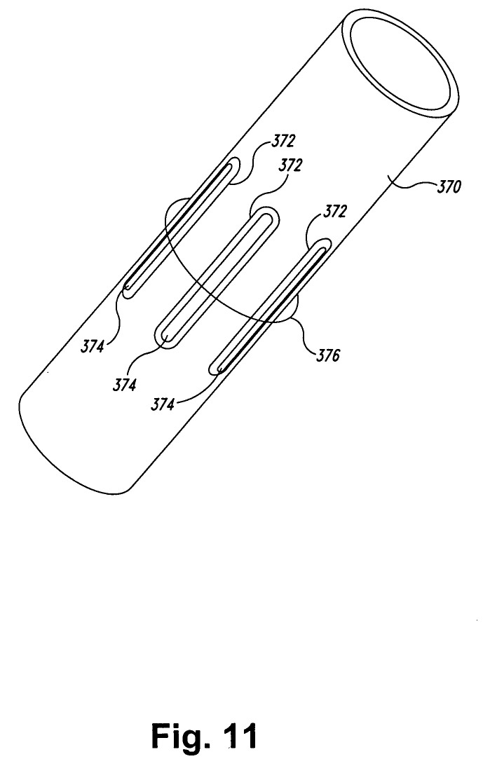

FIG. 10 is an exploded view of a one-part trocar for use in laparoscopic procedures. The trocar includes a single cannula 360, which includes at least one longitudinal slot 362. The slot is configured to be sealed by insert 364. In the illustrated embodiment, insert 364 is composed of a flexible material (e.g., silicone), and includes projection 366, which is arranged to fit into slot 362. In one method of use, this trocar may be inserted into a patient and used for insufflation, with projection 366 inserted into slot 362 to form a seal. When surgery is completed and insufflation is no longer required, insert 364 may be peeled back, allowing the surgeon to access the fascia via slot 362. In some embodiments, cannula 360 may be transparent in at least the region of slot 362, for example to aid the surgeon in visualizing the fascia.

FIG. 11 illustrates a one-part trocar with externally mounted tissue anchors. The trocar includes cannula 370, which in some embodiments may be fully or partially transparent. The cannula includes longitudinal recesses 372, each of which contains a tissue anchor 374. In the illustrated embodiment, the anchors are shape-memory wires of the type depicted in FIG. 8. Before deployment, the anchors are substantially straight and contained in recesses 372. In the illustrated embodiment, an optional looped filament 376 (e.g., a suture, cord, or wire) surrounds the cannula 370 and anchors 374. (For clarity, filament 376 is shown loosely wrapped about cannula 370; in some embodiments, the filament may be tightly wrapped, for example to secure the anchors in recesses 372.) In one method of use, the trocar is positioned for laparoscopic surgery. Before, during, or after surgery, the shape-memory wires of tissue anchors 374 are positioned adjacent to a fascia and activated, for example by applied heat. Upon activation, the anchors bend to pierce the tissue as shown in FIG. 8. The cannula 370 may then be withdrawn from the patient, leaving behind anchors 374 and filament 376. The filament 376 may be tightened to close the incision through which the cannula was inserted (for example, the filament may be a shape-memory suture which is induced to contract), or another suture or other approximating member may be used to secure the anchors together. In other embodiments, rather than shape-memory wires, other types of tissue anchors may be mounted on the exterior of cannula 370. In some of these embodiments, the cannula may include mechanical or other deployment mechanisms. For example, in one embodiment, split-ring anchors may be opened and restrained under tension in the recesses 372. The tension may be released, for example by removing a restraining member, allowing the anchors to pierce the tissue and close. In another embodiment, a portion of a multipart anchor (e.g., the anchors illustrated in FIG. 5, or FIG. 6, such as tissue-adherent portion 242 of anchor 240) may be deployed on the exterior of cannula 370.

In any of the herein-described trocar arrangements, an appropriate anchor deployment device may be used to place the anchors in the fascia. For example, U.S. Pat. No. 5,392,978, which is incorporated by reference herein, describes a surgical stapler for endoscopic use which crimps staples to secure them in tissue. An analogous deployment mechanism may be used to deliver tissue anchors through the longitudinal slots of the trocars illustrated herein. In other embodiments, surgical staplers such as those described in copending and commonly owned U.S. patent application Ser. No. 11/804,219, filed May 16, 2007, and entitled “STEERABLE SURGICAL STAPLER,” which is incorporated by reference herein, may be used to access tissue through the trocars. In addition, it will be understood that while the openings of the herein-illustrated trocars are configured as longitudinal slots, other geometries that allow access to the fascia will be apparent to those of ordinary skill in the art and are within the scope of the appended claims.

In general, the anchors, couplers, traction members, securing members, tensioning members, stabilizing members, and other components described herein may be adjustable or selectively controlled, for example to loosen tension as a joint heals and becomes more flexible or to permit expansion of skin prior to reconstructive surgery or removal for a graft. In particular, any of these components may form a part of or be configured to cooperate with the adjustable implants described in co-pending and commonly owned U.S. application Ser. Nos. 11/710,591, filed Feb. 22, 2007 and entitled, “CODED-SEQUENCE ACTIVATION OF SURGICAL IMPLANTS,” and 11/710,592, filed Feb. 22, 2007 and entitled, “CODED-SEQUENCE ACTIVATION OF SURGICAL IMPLANTS,” both of which are incorporated by reference herein. Any of these components may also be controllable by changing shape or conformation so that such change results in the approximation of surfaces attached to selected anchors, for example via the use of temperature-sensitive, light-sensitive (e.g., ultraviolet light-sensitive), touch-sensitive, elastomeric (e.g., an elastomer that is configured to secure each anchor and can reconfigure in a way to approximate surfaces attached to the anchors), or remotely controllable mechanisms.

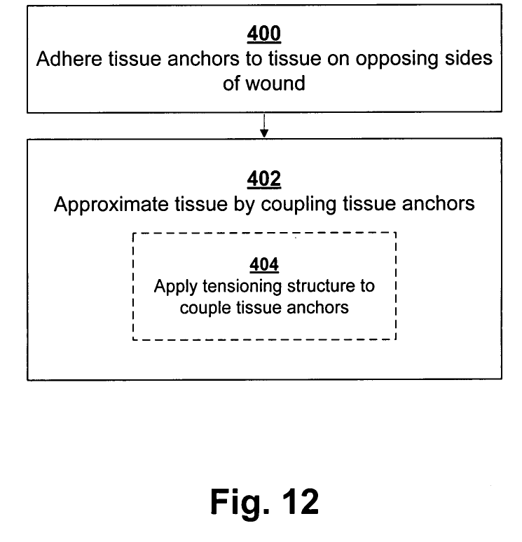

FIG. 12 is a flow chart illustrating a method of closing a wound. The method includes adhering tissue anchors (e.g., anchors such as but not limited to those described in FIG. 2, FIG. 5, or FIG. 6) to tissue on opposing sides of a wound, 400, and approximating the tissue by coupling the tissue anchors, 402. For example, the tissue anchors may be coupled via a tensioning element such as a suture, 404.

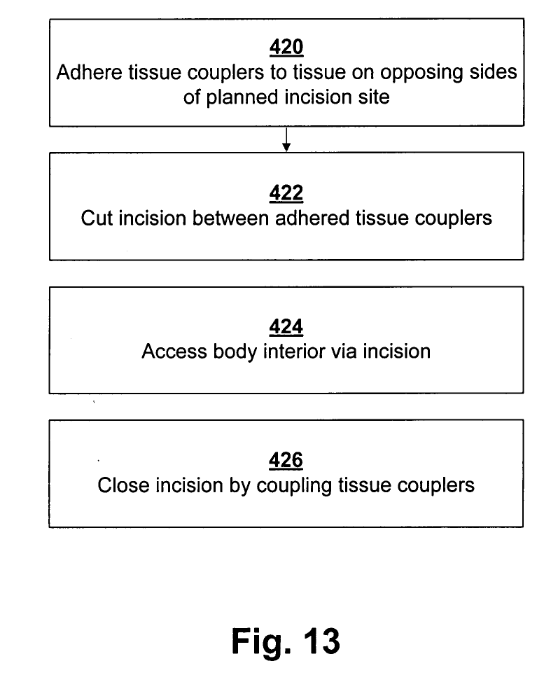

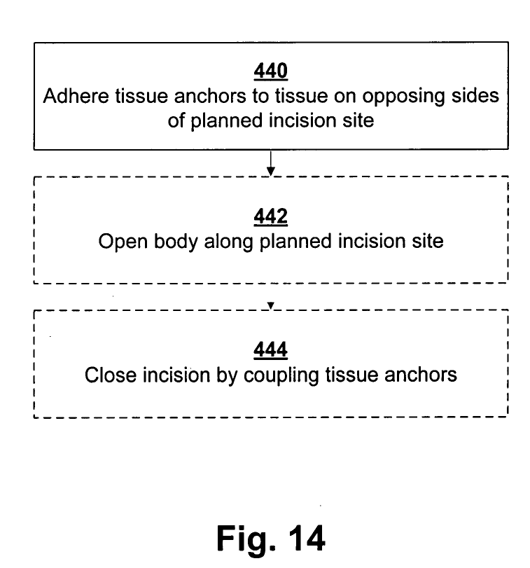

FIG. 13 is a flow chart illustrating a method of performing surgery. The method includes adhering tissue couplers (e.g., couplers such as but not limited to those described in FIG. 2, FIG. 5, or FIG. 6) to tissue on opposing sides of a planned incision site, 420, cutting an incision between the adhered tissue couplers, 422, accessing the interior of the body via the incision (e.g., to perform a surgical procedure), 424, and closing the incision by coupling the tissue couplers, 426. The incision may be, for example, a straight incision, a curved incision, or a round incision (e.g., a round cut such as that made by a trocar). In some embodiments, couplers may be coupled together manually, while in other embodiments, couplers may be coupled together automatically. In some embodiments, the surgery may be endoscopic.

FIG. 14 is a flow chart illustrating a method of preparing a body for surgery. The method includes adhering tissue anchors to tissue, 440, on opposing sides of a planned incision site. The method may optionally also include opening the body along the planned incision site, 442, or closing the incision by coupling the tissue anchors, 444. The incision may be, for example, a straight incision, a curved incision, or a round incision (e.g., a round cut such as that made by a trocar).

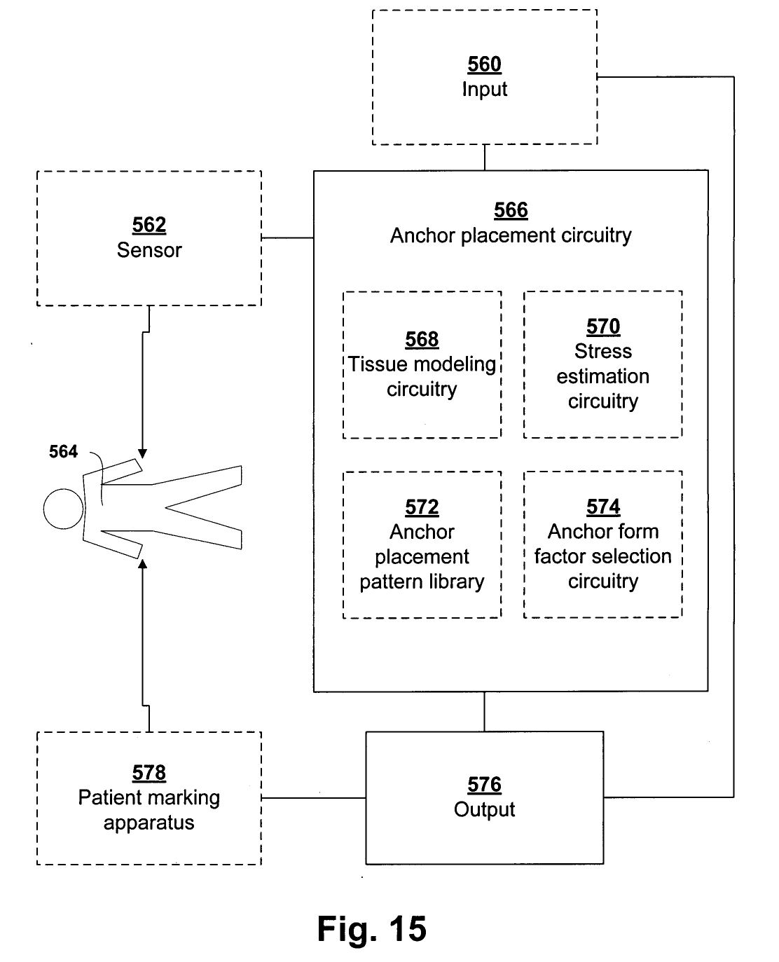

FIG. 15 illustrates a system for determining placement of tissue anchors (or other suture attachments) for closing an incision. The system may include an input device 560 (e.g., a mouse, keyboard, touchscreen, or other machine input system), configured to allow a surgeon to specify a surgery type or an incision location. It may further include a sensor 562 that measures one or more physiological parameters of a patient 564 upon whom surgery will be performed. For example, the sensor 562 may include an imaging device that maps the position of organs or other physiological structures that may be taken into account in closing an incision, or it may be a reader (e.g., an optical reader) that senses a planned incision location that a surgeon has marked on the body of patient 564. The sensor 562 may either directly recognize a body structure, or it may recognize an agent that has been introduced into the body to tag or otherwise identify a cell, organ, or other physiological structure. By way of non-limiting example, the sensor may sense a tumor-binding agent to determine the size and morphology of a tumor. This geometrical information could be used to plan the surgery as discussed elsewhere herein. The input device 560 or the sensor 562 (collectively, “input circuitry”) may communicate information about the body of patient 564 or about the planned surgery to anchor placement circuitry 566. Anchor placement circuitry 566 may include various subcircuits or subroutines, including but not limited to tissue modeling circuitry 568, stress estimation circuitry 570, anchor placement pattern library 572, or anchor form factor selection circuitry 574.

Tissue modeling circuitry 568 may include circuitry configured to build a computer-based model (e.g., a finite element model or an analytical model) of the tissue of the patient 564, for example including specific measurements of sensor 562 or physiological or other parameters specified using input device 560. This computer-based model may be used to determine suggested placement for tissue anchors, for example by calculation of the expected response of tissue to particular anchor configurations, or by application of stored heuristic rules for expected tissue response. Stress estimation circuitry 570 may be configured to determine expected stresses on anchors or on tissue for particular anchor configurations, or it may include optimizing circuitry designed to determine an optimum anchor configuration for a specified design goal. Anchor placement pattern library 372 may include stored configurations of anchors that have been specified by an operator, previously calculated, or otherwise determined. Other portions of the anchor placement circuitry 566 (e.g. tissue modeling circuitry 568 or stress estimation circuitry 570) may use the anchor placement pattern library 572 to generate initial placement patterns for calculation, including as a starting point for optimization routines. Anchor form factor selection circuitry 574 may store information about the different form factors of different anchors (such as but not limited to those described herein, e.g., in FIG. 2, FIG. 5, or FIG. 6), and may further include information about available sizes and mechanical performance of different anchors. It may further include circuitry configured to select a suggested anchor or group of anchors for the particular surgery planned for patient 564.

As illustrated in FIG. 15, the system further includes an output device 576 (e.g., a monitor, a printer, a bar code printer, or a controller for a patient marking apparatus 578), which may produce a machine-readable or a human-readable output. This output may include calculated anchor placement patterns, tissue responses, anchor stresses, anchor form factors, or other data relevant for placement of anchors during surgery. Output may be iterative or interactive, so that a user specifying input via input device 560 may modify input or specify additional inputs in response to output received via output device 576. For example, output device 576 may output a selection of anchor placement patterns from anchor placement pattern library 572, and a user may select from among these patterns using input device 560. Once an anchor placement pattern has been established by anchor placement circuitry 566, output device 576 may display the selection. For example, it may visually display the determined anchor placement on a screen, or it may pass data or control instructions to a patient marking device 578, which may temporarily or permanently mark desired anchor placement directly on the patient 564, or on a tape or other stabilizing member configured to maintain relative anchor locations for attachment to the patient 564. In other embodiments, the patient marking device may place anchors on a stabilizing member for application to a patient 564, or apply anchors directly to a patient.

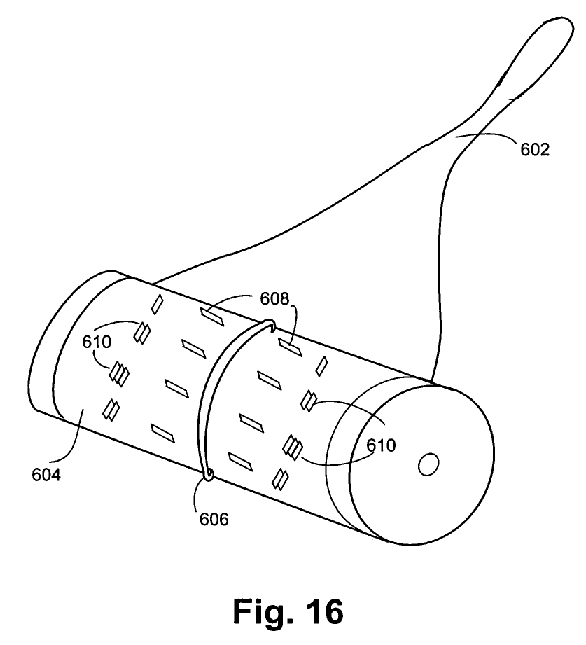

FIG. 16 illustrates a marking roller for use before or during surgery to place fiducial markings to assist the surgeon in closing an incision. The roller includes handle 602 and roller 604. Roller 604 may be pre-inked with any of a variety of inks suitable for marking the skin, such as but not limited to the inks described in U.S. Pat. Nos. 6,056,737 and 6,972,022, which are incorporated herein by reference. As shown, roller 604 includes a central rib 606 which is configured to produce a substantially continuous line as the roller 604 is rolled across the skin. On opposing sides of central rib 606, protrusions 608 are arranged to produce fiducial marks that will aid the surgeon in maintaining alignment of the skin as the incision is closed. Further protrusions 610 are configured to place additional markings that may act as labels for the fiducial marks produced by protrusions 608. In the illustrated embodiment, these additional markings serve to identify pairs of fiducial marks to prevent a mismatch error as the fiducials are aligned during closing. In other embodiments, additional markings may also indicate what type of anchor is to be used at the fiducial mark, or other information about the surgery. In some embodiments, the fiducial marks produced by protrusions 608 or 610 may be human-readable, while in other embodiments, they may be machine-readable. The fiducial marks may simply facilitate alignment, or, in other embodiments, they may identify surgical attachment points where tissue anchors, staples, stitches, or other attachment devices may be used to close an incision or otherwise attach tissue. In some embodiments, the placement of fiducial marks may be standardized in a general-purpose roller, while in other embodiments, a customized roller 604 may be constructed for a patient, for example by using known rapid prototyping techniques.

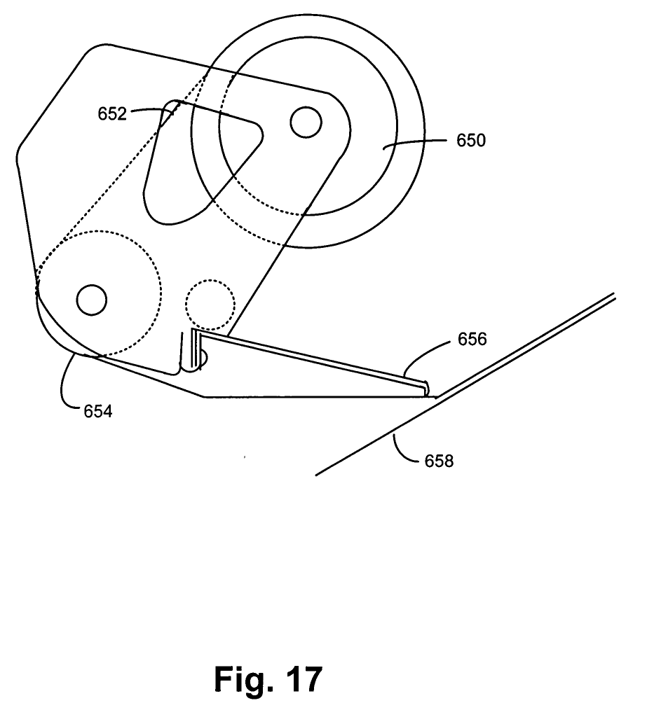

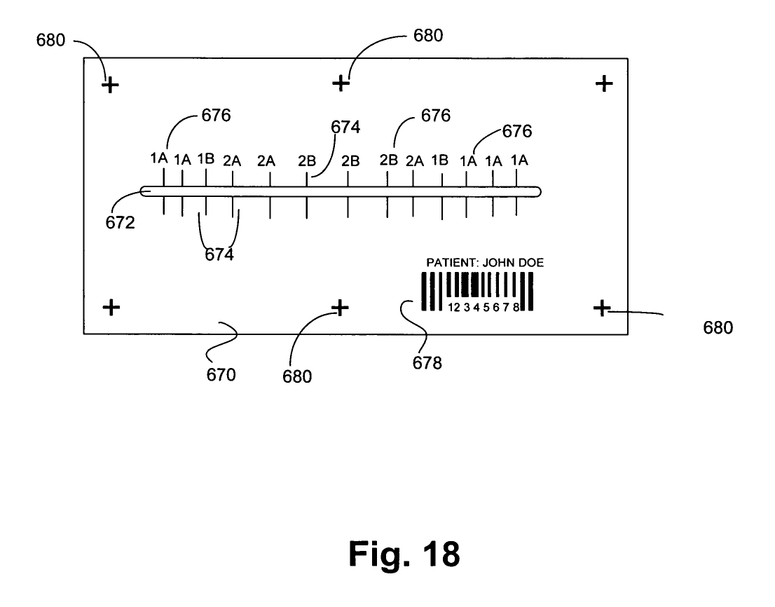

FIG. 17 illustrates a tape dispenser configured to dispense a tape for attachment to a surgical patient (e.g., a human, other mammal, or other animal). The tape may be, in one embodiment, the stabilizing member 102 illustrated in FIG. 4, which includes anchors mounted thereon. Another suitable tape is illustrated in FIG. 18, as described below. (As used herein, the term “tape” includes substrates that are largely, although not necessarily entirely, two-dimensional in nature. Tapes may include adhesive or other structures for affixation to a patient, and may be flexible or rigid.) The illustrated tape dispenser includes a dispensing roller 650 on which a loaded tape 652 is wound, a tensioning roller 654 that maintains a suitable tension in tape 652 during dispensing, and an applicator 656 that smoothes dispensed tape along the skin 658. In some embodiments, the applicator 656 is arranged so that the skin 658 can easily be seen by the surgeon as the tape 652 is applied. The dispenser may further include a handle or other grasping mechanism (not shown).

FIG. 18 illustrates a tape suitable for loading in the dispenser illustrated in FIG. 17. The tape includes an adhesive base 670, which includes an elongated opening 672 which serves as a fiducial marking an incision location. In other embodiments (not shown), the tape may include a marking such as a substantially continuous line indicating an incision location instead of an opening. The tape further includes a number of fiducial marks 674, each of which indicates a predetermined attachment point for closure of the incision. In the illustrated embodiment, the fiducial marks are located at positions opposing one another across opening 672. In other embodiments, fiducials may not appear on both sides of the incision-indicating fiducial (if present). In still other embodiments, fiducials may not be aligned on opposing sides of the incision-indicating fiducial (if present). For example, in certain types of surgery (e.g., cosmetic surgery), it may be desirable to reattach skin in a different configuration from the configuration before opening. In some embodiments, the locations of fiducial marks 674 may be determined by techniques described herein such as tissue modeling or stress estimation, or may be printed on the adhesive tape base 670 by an output device as described herein in connection with FIG. 15.

In the illustrated embodiment, each pair of fiducial marks 674 is labeled with a human-readable label 676. As illustrated, a simple code identifies the type of surgical attachment (e.g., the size or material of a tissue anchor as described herein) that is to be used at each point. In other embodiments, the labels 676 may contain more extensive information about the fiducials, or may be machine-readable. Although the illustrated embodiment includes a fiducial mark 674 and label 676 for each contemplated attachment point, other embodiments may include more or fewer fiducial marks or labels.

The illustrated tape further includes an optional patient identifier 678, which includes both a human-readable patient name and a machine-readable bar code. In other embodiments (not shown), the tape may further include more detailed information, including without limitation surgery type, surgery location, planned anesthesia parameters, allergy information, or other information about the patient or the planned surgery.

In addition, the illustrated tape includes registration marks 680. These marks may be used to align the tape in the correct location on the patient, for example by placing them on known anatomical features or on indicia previously placed on the skin.

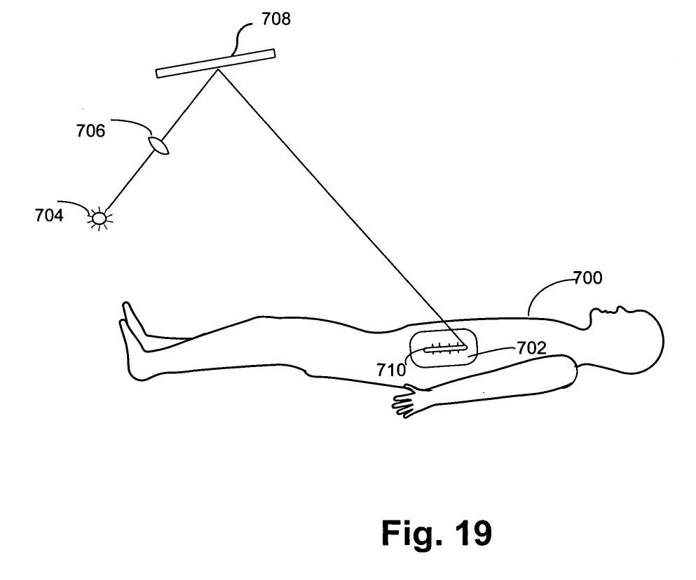

FIG. 19 illustrates a surgical tool for placement of fiducial marks on a body 700 of a person. An energy-responsive layer 702 is placed on the body, which is then exposed to energy from an energy source 704. In some embodiments, the energy-responsive layer 702 may be in the form of a sheet affixed to the body, while in other embodiments, it may be a liquid, gel, lotion, or cream that is applied to the body (which, in some embodiments, may be allowed to dry or may be absorbed into the skin). In some embodiments, the energy-responsive layer 702 may be photosensitive, and the energy source 704 may be a light source such as a laser. In other embodiments, the energy source may be, for example, a particle beam generator, a magnetic or electric field generator, or an electromagnetic beam generator. In the illustrated embodiment, optional beam-directing elements of a lens 706 and a mirror 708 are interposed in the optical path between the energy source 704 and the energy-responsive layer 702. Beam-directing elements may include, without limitation, lenses, splitters, masks, polarizers, mirrors, electromagnetic fields, diffractive elements, or refractive elements. While lens 706 has been depicted in the traditional shape of a refractive lens, other types of lenses may also be used to direct energy emanating from the energy-source. Examples of suitable energy sources and energy-responsive layers are described in commonly-assigned and copending U.S. patent application Ser. Nos. 11/143,116, filed Jun. 2, 2005, entitled “PHOTOPATTERNING OF SKIN” and published as U.S. Patent Publication No. 2006/0276859 A1, and 11/198,910, filed Aug. 5, 2005 and entitled “HOLOGRAPHIC TATTOO,” both of which are incorporated by reference herein.

Exposure of energy-responsive layer 702 to energy from energy source 704 forms at least one fiducial mark 710 indicative of a surgical plan. For example, the fiducial mark 710 may indicate a planned incision location, one or more planned locations for surgical closure elements (e.g., a tissue anchor or a suture), or identifying information for surgical closure elements. In some embodiments, the fiducial mark may specify three-dimensional surgical information such as planned incisions or attachment points in successive tissue layers. In some such embodiments, this three-dimensional information may be conveniently conveyed using the holographic “tattoos” described in U.S. patent application Ser. No. 11/198,910. The surgical tool may further include attachment placement circuitry (not shown) such as that described in connection with FIG. 15, in which case a controller may be configured to direct energy from energy source 704 in accordance with planned attachment points determined by the attachment placement circuitry.

Various embodiments of tools and systems for placing fiducial markings on a patient have been described herein. It will be understood that the selection of a particular tool is within the competence of one of ordinary skill in the art; who will further understand that certain features of a tool described herein may be applied to another tool. By way of non-limiting example, the three-dimensional surgical information described in connection with FIG. 19 may readily be provided by a marking roller (such as the one shown in FIG. 16), or a tape (such as the one shown in FIG. 18). Other features of the different tools, systems, and methods herein described may be similarly applied from one embodiment to another. Further, while fiducial markings have largely been described herein as being placed on the skin, it will be understood that fiducial markings may also be placed on other tissues according to the principles described herein, such as but not limited to muscles, bone, organs, or connective tissues.

In a general sense, those skilled in the art will recognize that the various aspects described herein which can be implemented, individually or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes or devices described herein, or a microprocessor configured by a computer program which at least partially carries out processes or devices described herein), electrical circuitry forming a memory device (e.g., forms of random access memory), or electrical circuitry forming a communications device (e.g., a modem, communications switch, or optical-electrical equipment). Those having skill in the art will recognize that the subject matter described herein may be implemented in an analog or digital fashion or some combination thereof.

Those having skill in the art will recognize that the state of the art of circuit design has progressed to the point where there is typically little distinction left between hardware and software implementations of aspects of systems. The use of hardware or software is generally a design choice representing tradeoffs between cost, efficiency, flexibility, and other implementation considerations. Those having skill in the art will appreciate that there are various vehicles by which processes, systems or other technologies involving the use of logic or circuits can be effected (e.g., hardware, software, or firmware), and that the preferred vehicle will vary with the context in which the processes, systems or other technologies are deployed. For example, if an implementer determines that speed is paramount, the implementer may opt for a mainly hardware or firmware vehicle. Alternatively, if flexibility is paramount, the implementer may opt for a mainly software implementation. In these or other situations, the implementer may also opt for some combination of hardware, software, or firmware. Hence, there are several possible vehicles by which the processes, devices or other technologies involving logic or circuits described herein may be effected, none of which is inherently superior to the other. Those skilled in the art will recognize that optical aspects of implementations may require optically-oriented hardware, software, and or firmware.

It will be understood by those within the art that, in general, terms used herein, and especially in the appended claims are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of introductory phrases such as “at least one” or “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “an anchor” should typically be interpreted to mean “at least one anchor”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two anchors,” or “a plurality of anchors,” without other modifiers, typically means at least two anchors). Furthermore, in those instances where a phrase such as “at least one of A, B, and C,” “at least one of A, B, or C,” or “an [item] selected from the group consisting of A, B, and C,” is used, in general such a construction is intended in the sense one having skill in the art would understand the convention (e.g., any of these phrases would include but not be limited to systems that have A alone, B alone, C alone, A and B together, A and C together, B and C together, or A, B, and C together). It will be further understood by those within the art that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A system for planning placement of tissue anchors during a surgery, comprising:
input circuitry configured to receive a signal including information relating to a planned surgery on a body of an animal; and
anchor placement circuitry configured to use the received signal to determine preferred placement of tissue anchors for the surgery.

2. The system of claim 1, wherein the input circuitry includes a sensor configured to measure a physiological parameter of the animal.

3-9. (canceled)

10. The system of claim 1, wherein the signal includes a physiological parameter of the animal.

11. (canceled)

12. The system of claim 1, wherein the anchor placement circuitry includes tissue modeling circuitry.

13. The system of claim 12, wherein the tissue modeling circuitry is configured to build a virtual model of at least a portion of the body.

14-15. (canceled)

16. The system of claim 1, wherein the anchor placement circuitry includes stress estimation circuitry.

17-19. (canceled)

20. The system of claim 1, wherein the anchor placement circuitry includes an anchor placement pattern library.

21-23. (canceled)

24. The system of claim 1, wherein the anchor placement circuitry includes anchor form factor selection circuitry.

25-28. (canceled)

29. The system of claim 1, further comprising an output configured to display the determined preferred placement of tissue anchors.

30-31. (canceled)

32. The system of claim 29, wherein the output includes a patient marking device.

33-35. (canceled)

36. The system of claim 29, wherein the output includes a printer that marks a tape with fiducial marks indicating the preferred placement of tissue anchors, wherein the tape is configured to be attached to the body of the animal.

37-39. (canceled)

40. A tool for placement of fiducial marks on a body of an animal, comprising:
a marking roller configured to be rolled along the surface of the body of the animal, wherein the marking roller includes a fiducial-marking surface configured to make at least one fiducial mark on the body as the fiducial-marking surface comes in contact with the surface of the body; the at least one fiducial mark on the body indicating locations for a surgical incision and for a surgical closure element.

41-42. (canceled)

43. The tool of claim 40, wherein the at least one fiducial mark includes a substantially continuous line along a path followed by the roller.

44. The tool of claim 43, wherein the substantially continuous line indicates the planned location of the surgical incision.

45. The tool of claim 43, wherein the at least one fiducial mark includes an alignment mark offset from the substantially continuous line.

46. The tool of claim 45, wherein the alignment mark indicates the planned location of a surgical closure element.

47. The tool of claim 40, wherein the roller includes a cutting edge configured to cut tissue along a cutting path.

48. The tool of claim 47, wherein the cutting path indicates the location of the surgical incision.

49-52. (canceled)

53. The tool of claim 40, wherein the tool is configured to make the at least one fiducial mark on the animal's skin.

54. The tool of claim 40, wherein the tool is configured to make the at least one fiducial mark on a tissue of the animal selected from the group consisting of bone, muscle, organs, and connective tissue.

55. A surgical tool for placement of fiducial marks on a body of an animal, comprising:
a tape dispenser configured to dispense a tape configured for attachment to the body of the animal, wherein the tape includes at least one fiducial mark thereon that indicates a surgical plan.

56. The tool of claim 55, wherein the tape includes an adhesive layer configured to attach the tape to the body of the animal.

57. The tool of claim 55, wherein the at least one fiducial mark identifies an incision location.

58. The tool of claim 55, wherein the tape includes an opening for an incision.

59-60. (canceled)

61. The tool of claim 55, wherein the tape dispenser comprises a roller loaded with the marked tape.

62. (canceled)

63. The tool of claim 55, further comprising a printer configured to place the at least one fiducial mark on the tape.

64-67. (canceled)

68. A surgical tool for placement of fiducial marks on a body of an animal, comprising:
an energy source configured to induce a modification of an energy-responsive material on the body, wherein the modification forms at least one fiducial mark indicative of a surgical plan.

69. The tool of claim 68, wherein the at least one fiducial mark indicates an incision location.

70. The tool of claim 68, wherein the at least one fiducial mark indicates a location of a surgical closure element.

71. The tool of claim 68, further comprising:
identification circuitry configured to
identify an incision location for a surgical closure; and
determine an incision mark location for the at least one fiducial mark that characterizes the incision location for the surgical closure; and
controller circuitry configured to direct the energy source to modify the energy-responsive material in accordance with the determined incision mark location to form the at least one fiducial mark.

72-74. (canceled)

75. The tool of claim 68, wherein the energy source produces electromagnetic energy.

76-77. (canceled)

78. The tool of claim 68, wherein the energy source produces a particle beam.

79-84. (canceled)

85. A method of preparing a patient for surgery, comprising:
identifying at least one preferred attachment point for a surgical closure; and
placing at least one fiducial mark at each of the at least one preferred attachment points, wherein the at least one fiducial mark is machine-readable.

86. The method of claim 85, wherein placing the at least one fiducial mark includes placing the at least one fiducial mark with a marking roller.

87. The method of claim 86, further comprising constructing the marking roller in a configuration to place the at least one fiducial mark at each of the at least one preferred attachment points.

88. The method of claim 85, wherein placing the at least one fiducial mark includes adhering a stabilizing member to the patient, wherein the stabilizing member includes the at least one fiducial mark.

89. The method of claim 85, wherein placing the at least one fiducial mark includes:
applying an energy-sensitive material to the patient; and
exposing the energy-sensitive material to a spatially patterned energy flux that modifies the energy-sensitive material to produce the at least one fiducial mark.

90. The method of claim 85, wherein identifying at least one preferred attachment point for a surgical closure includes measuring a physiological parameter of the patient.

91. The method of claim 85, wherein identifying at least one preferred attachment point for a surgical closure includes constructing a virtual model of at least a portion of the patient.

92. (canceled)

93. The method of claim 85, wherein identifying at least one preferred attachment point for a surgical closure includes determining expected stresses on a surgical closure at the at least one preferred attachment point.

94-99. (canceled)

100. A method of preparing a patient for surgery, comprising:
identifying at least one preferred attachment point for a surgical closure; and
placing at least one fiducial mark on a stabilizing member configured to be adhered to the patient, wherein the at least one fiducial mark is placed to indicate the at least one preferred attachment point upon adhering the stabilizing member to the patient.

101. The method of claim 100, further comprising adhering the stabilizing member to the patient.

102-103. (canceled)

104. The method of claim 101, wherein adhering the stabilizing member to the patient includes loading the stabilizing member into a dispenser and dispensing the stabilizing member from the dispenser.

105-109. (canceled)

110. The method of claim 100, wherein placing the at least one fiducial mark on the stabilizing member includes printing the at least one fiducial mark on the stabilizing member.

111. The method of claim 100, wherein the stabilizing member includes at least one registration mark configured to identify a location for placement of the stabilizing member on the patient.

112-113. (canceled)

114. The method of claim 100, wherein the at least one fiducial mark includes three-dimensional surgical information.

115-116. (canceled)

117. A method of preparing the body of a patient for surgery, comprising:
identifying at least one preferred attachment point for a surgical closure;
applying an energy-responsive material to the body; and
exposing the energy-responsive material to a spatially patterned energy flux, wherein the spatially patterned energy flux modifies the energy-responsive material to form at least one fiducial mark placed to indicate the at least one preferred attachment point for the surgical closure.

118. The method of claim 117, wherein identifying at least one preferred attachment point for a surgical closure includes measuring a physiological parameter of the patient.

119-122. (canceled)

123. The method of claim 117, wherein the spatially patterned energy flux is electromagnetic radiation.

124. (canceled)

125. The method of claim 117, wherein the at least one fiducial mark includes three-dimensional surgical information.

126-129. (canceled)
